(19) United States
(12) Patent Application Publication
(10) Pub. No.: US 2022/0039770 A1
(43) Pub. Date: Feb. 10, 2022

(54) Systems and Methods for Identification of Pulmonary Conditions

(71) Applicant: The Board of Trustees of the Leland Stanford Junior University, Stanford, CA (US)

(72) Inventors: Husham Sharifi, Stanford, CA (US); Joe Hsu, Stanford, CA (US); Haiwei Henry Guo, Stanford, CA (US); Serena Yu-Ching Yeung-Levy, Stanford, CA (US); Seyyed Ali Mottaghi, Stanford, CA (US)

(73) Assignee: The Board of Trustees of the Leland Stanford Junior University, Stanford, CA (US)

(21) Appl. No.: 17/398,904

(22) Filed: Aug. 10, 2021

Related U.S. Application Data
(60) Provisional application No. 63/063,925, filed on Aug. 10, 2020.

(51) Int. Cl. A61B 6/00; A61B 6/03; G16H 30/40; G16H 15/00; G16H 50/20; G06N 20/10

(52) U.S. Cl. CPC A61B 6/5217; A61B 6/032; A61B 6/505; G06N 20/10; G16H 30/40; G16H 15/00; G16H 50/20; A61B 6/5223

(57) ABSTRACT

Systems and methods for identification of pulmonary conditions accordance with embodiments of the invention are illustrated. One embodiment includes a method for identifying pulmonary conditions in hematopoietic cell transplantation patients, include obtaining a computed tomography (CT) scan of a patient's lungs, calculating a plurality of parametric response mapping (PRM) metrics, providing the plurality of PRM metrics to a machine learning model, obtaining a classification of the CT scan as indicating whether or not the patient's lungs present with a pulmonary condition, and providing a report comprising the classification.

### CROSS-REFERENCE TO RELATED APPLICATIONS

The current application claims the benefit of and priority under 35 U.S.C. § 119(e) to U.S. Provisional Patent Application No. 63/063,925 titled “Machine Learning Algorithms to Differentiate Among Pulmonary Complications After Hematopoietic Cell Transplant”, filed Aug. 10, 2020. The disclosure of U.S. Provisional Patent Application No. 63/063,925 is hereby incorporated by reference in its entirety.

### FIELD OF THE INVENTION

The present invention generally relates to systems and methods for identifying pulmonary conditions, namely differentiation among pulmonary complications after hematopoietic cell transplantation (HCT).

### BACKGROUND

Hematopoietic cell transplantation (HCT), also referred to as hematopoietic stem-cell transplantation (HSCT) is the transplantation of multipotent hematopoietic stem cells most often used for the treatment of cancers, autoimmune diseases, and hereditary skeletal dysplasias. HCT is a dangerous procedure with a great number of complications, and therefore is often reserved for patients with life-threatening diseases. Especially in the case of allogenic transplantation, graft-versus host diseases is a major complication.

Support-vector machines (SVMs) are a class of machine learning model associated with learning algorithms that analyze data for classification and regression analysis. SVMs are traditionally supervised learning models, which require training on a set of training data in order to function. k-means clustering is a method of vector quantization that aims to partition n observations into k clusters in which each observation belongs to the cluster with the nearest mean, serving as a protype of the cluster.

### SUMMARY OF THE INVENTION

Systems and methods for identification of pulmonary conditions accordance with embodiments of the invention are illustrated. One embodiment includes a method for identifying pulmonary conditions in hematopoietic cell transplantation patients, include obtaining a computed tomography (CT) scan of a patient's lungs, calculating a plurality of parametric response mapping (PRM) metrics, providing the plurality of PRM metrics to a machine learning model, obtaining a classification of the CT scan as indicating whether or not the patient's lungs present with a pulmonary condition, and providing a report comprising the classification.

In another embodiment, the PRM metrics include classifications of voxels in the CT scan as presenting with one of: normal lung parenchyma, functional small airway disease, emphysema, and parenchymal disease.

In a further embodiment, further including providing at least one metric describing an erector spinae muscle of the patient (ES) to the machine learning model.

In still another embodiment, wherein the at least one metric describing the ES is calculated by locating bones within coronal slices of the CT scan, identifying a coronal slice of the CT scan that contains an image of a spine of the patient, locating the bottom-most rib of the patient in the coronal slice, providing an axial slice of the CT scan at the location of the bottom-most rib to a mask model, and obtaining a mask describing the area of the axial slice that contains an image of the ES from the mask model.

In a still further embodiment, the at least one metric is cross-sectional area of the ES, and the method further comprising calculating the cross-sectional area of the ES as the area of the axial slice containing the image of the ES according to the mask.

In yet another embodiment, the at least one metric is density of the ES, and the method further comprising calculating the density as the average Housenfield unit value of voxels in the axial slice containing the image of the ES according to the mask.

In a yet further embodiment, the method further includes discarding the top half of the CT scan.

In another additional embodiment, the machine learning model is a support vector machine.

In a further additional embodiment, the pulmonary condition is bronchiolitis obliterans syndrome.

In another embodiment again, a device for identifying pulmonary conditions in hematopoietic cell transplantation patients, including a processor, and a memory, the memory containing a pulmonary condition identification application directs the processor to obtain a computed tomography (CT) scan of a patient's lungs, calculate a plurality of parametric response mapping (PRM) metrics, provide the plurality of PRM metrics to a machine learning model, obtain a classification of the CT scan as indicating whether or not the patient's lungs present with a pulmonary condition, and provide a report comprising the classification.

In a further embodiment again, the PRM metrics include classifications of voxels in the CT scan as presenting with one of: normal lung parenchyma, functional small airway disease, emphysema, and parenchymal disease.

In still yet another embodiment, the pulmonary condition identification application further directs the processor to provide at least one metric describing an erector spinae muscle of the patient (ES) to the machine learning model.

In a still yet further embodiment, in order to calculate the at least one metric describing the ES, the pulmonary condition identification application further directs the processor to locate bones within coronal slices of the CT scan, identify a coronal slice of the CT scan that contains an image of a spine of the patient, locate the bottom-most rib of the patient in the coronal slice, provide an axial slice of the CT scan at the location of the bottom-most rib to a mask model, and obtain a mask describing the area of the axial slice that contains an image of the ES from the mask model.

In still another additional embodiment, the at least one metric is cross-sectional area of the ES, and the pulmonary condition identification application further directs the processor to calculate the cross-sectional area of the ES as the area of the axial slice containing the image of the ES according to the mask.

In a still further additional embodiment, the at least one metric is density of the ES, and the pulmonary condition identification application further directs the processor to calculate the density of the ES as the average Housenfield unit value of voxels in the axial slice containing the image of the ES according to the mask.

In still another embodiment again, the pulmonary condition identification application further directs the processor to discard the top half of the CT scan.

In a still further embodiment again, the machine learning model is a support vector machine.

In yet another additional embodiment, the pulmonary condition is bronchiolitis obliterans syndrome.

In a yet further additional embodiment, a method for identifying pulmonary conditions in hematopoietic cell transplantation patients, including obtaining a computed tomography (CT) scan of a patient's lungs, calculating a plurality of parametric response mapping (PRM) metrics, calculating a density and a cross-sectional area of an erector spinae muscle of the patient at a level of a T12 vertebra of the patient, providing the plurality of PRM metrics, the density, and the cross-sectional area to a machine learning model, obtaining a classification of the CT scan as indicating whether or not the patient's lungs present with a pulmonary condition, and providing a report comprising the classification.

In yet another embodiment again, the method further includes locating bones within coronal slices of the CT scan, identifying a coronal slice of the CT scan that contains an image of a spine of the patient, locating the bottom-most rib of the patient in the coronal slice, providing an axial slice of the CT scan at the location of the bottom-most rib to a mask model, and obtaining a mask describing the area of the axial slice that contains an image of the erector spinae from the mask model, calculating the cross-sectional area of the erector spinae muscle as the area of the axial slice containing the erector spinae according to the mask, and calculating the density of the erector spinae muscle as the average Housenfield unit value of voxels in the axial slice containing the erector spinae according to the mask.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a system diagram for a pulmonary condition identification system in accordance with an embodiment of the invention.

FIG. 2 is a block diagram of a pulmonary condition identifier in accordance with an embodiment of the invention.

FIG. 3 is a flow chart illustrating a process for identifying pulmonary conditions in accordance with an embodiment of the invention.

FIG. 4 is a flow chart illustrating a process for calculating the cross-sectional area and density of the erector spinae in accordance with an embodiment of the invention.

### DETAILED DESCRIPTION

Turning now to the drawings, systems and methods for identification of pulmonary conditions are described. Hematopoietic cell transplantation (HCT) is a lifesaving procedure that is associated with a number of significant risks. For example, pulmonary complications, including infections, are highly prevalent in patients after HCT with chronic graft-vs-host disease. However, the comorbidity of diseases can make the diagnosis of early lung graft-vs-host disease (bronchiolitis obliterans syndrome, BOS) challenging. Systems and methods described herein are able to identify pulmonary complications using computed tomography (CT) scans of patient's lungs in a qualitative manner.

In many embodiments, a machine learning model such as (but not limited to) a support vector machine (SVM) is provided with pulmonary parametric response mapping (PRM) metrics derived from the CT scan of the patient and provides a classification of the patient's lungs as suffering from BOS. However, as can be readily appreciated, alternate machine learning models can be used as appropriate to the requirements of specific applications of embodiments of the invention. Further, different pulmonary conditions can be identified depending on the training data provided to the machine learning model. Pulmonary condition identification systems are described in further detail below.

Pulmonary Condition Identification Systems

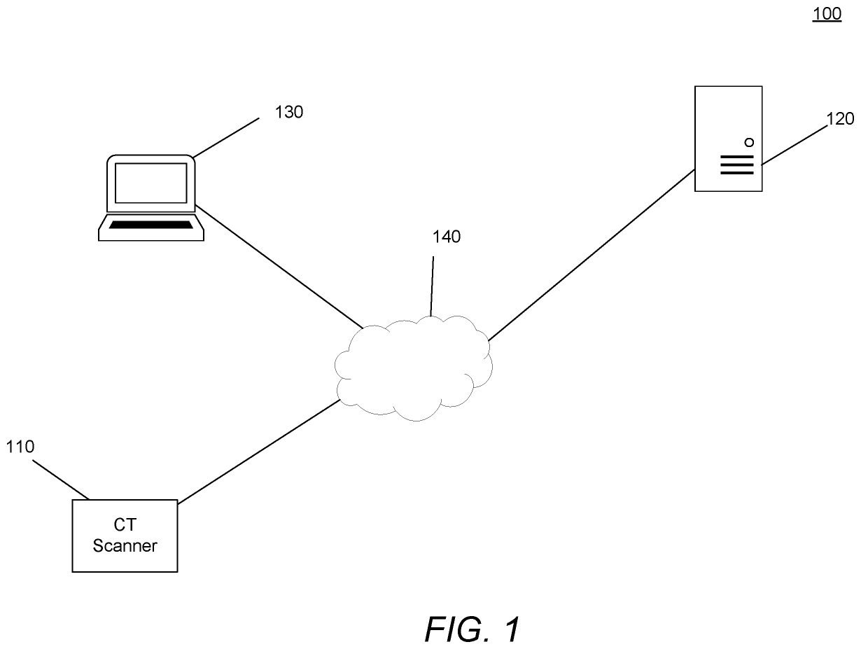

Turning now to FIG. 1, a system for identifying pulmonary conditions in accordance with an embodiment of the invention is illustrated. System 100 includes a CT scanner 110, a pulmonary condition identifier 120, and an interface device 130. In many embodiments, the pulmonary condition identifier is a personal computer, however any number of different types of computing device such as (but not limited to) servers, smart phones, cloud computing clusters, tablet computers, and/or any type of computing device can be used as appropriate to the requirements of specific applications of embodiments of the invention. The interface device is any device capable of providing display of the output of the pulmonary condition identifier and/or controlling the operation of the pulmonary condition identifier. In various embodiments, the interface device is a separate computing device, however the interface device may be incorporated into and/or be a peripheral device connected to the pulmonary condition identifier.

CT scanners, pulmonary condition identifiers, and interface devices communicate over a network 140. In many embodiments, the network 140 is the Internet. However, network 140 can be made up of local area networks, intranets, wide area networks, peer-to-peer networks, and/or any other network and/or combination of networks as appropriate to the requirements of specific applications of embodiments of the invention. Furthermore, as can be readily appreciated, while a specific system architecture is illustrated in FIG. 1, any number of architectures can be used as appropriate to the requirements of specific applications of embodiments of the invention, such as (but not limited to) those that utilize different radiological imaging modalities capable of producing data that can be processed to provide PRM metrics.

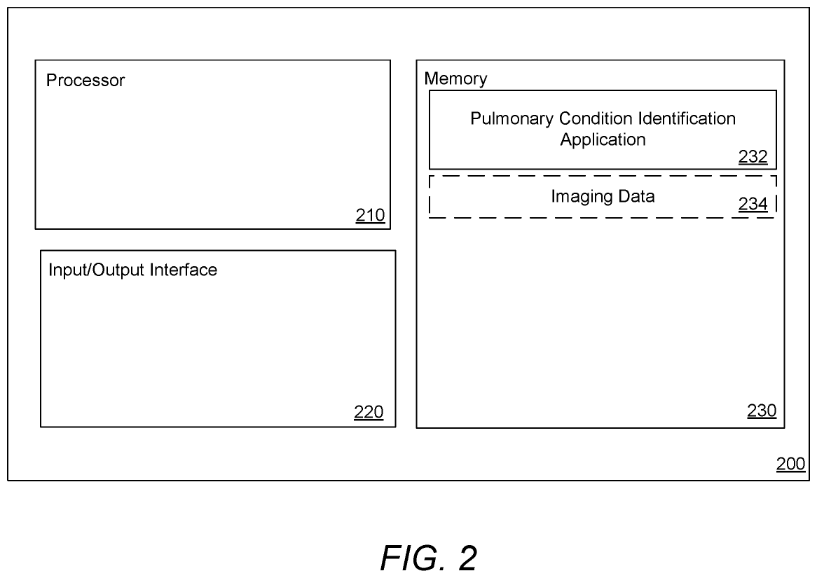

Turning now to FIG. 2, a block diagram for a pulmonary condition identifier in accordance with an embodiment of the invention is illustrated. Pulmonary condition identifier 200 includes a processor 210. Processor 210 can be any logic processing circuitry capable of performing crowdsourced machine learning processes. In many embodiments, processors are central processing units (CPUs), graphics processing units (GPUs), field-programmable gate arrays (FPGAs), application-specific integrated circuits (ASICs), and/or any other logic circuit and/or combination thereof as appropriate to the requirements of specific applications of embodiments of the invention.

Pulmonary condition identifier 200 further includes an input/output (I/O) interface 220. I/O interfaces are capable of transmitting data from crowdsourced machine learning devices to crowdsourcing platforms and/or any other computing device as appropriate to the requirements of specific applications of embodiments of the invention. Pulmonary condition identifier 200 further includes a memory 230 which contains a pulmonary condition identification application 232. Pulmonary condition identification applications are capable of directing the processor to carry out various pulmonary condition identification processes which are discussed in further detail below. In many embodiments, the memory 230 further includes imaging data produced by a CT scanner describing a CT scan of a patient's lungs. While a specific architecture for a pulmonary condition identifier is illustrated in FIG. 2, any number of different computational architectures can be used as appropriate to the requirements of specific applications of embodiments of the invention.

### Pulmonary Condition Identification Processes

Pulmonary condition identification processes enable the identification of pulmonary diseases within CT scans of patient's lungs. In many embodiments, a machine learning model such as a SVM is trained using a training data set of PRM metrics derived from a set of CT scans which have been annotated with the true condition of the patient of which each CT scan was taken. Subsequent to training, CT scans can be taken of new patients and PRM metrics can be derived, which in turn can be provided to the trained machine learning model to identify pulmonary conditions. In many embodiments, the machine learning model is trained to identify a specific pulmonary condition, e.g. BOS. In this case, multiple different models can be trained and utilized to identify different pulmonary conditions.

In many embodiments, PRM involves obtaining a biphasic CT of a patient (inspiration and expiration), processing the CT scan to align the inspiration and expiration phases so they share the same spatial geometry (each 1 Housenfield unit (HU) voxel at the inspiration phase corresponds to a 1 HU voxel in the expiration phase), and classifying each voxel into discrete zones as containing normal lung parenchyma, functional small airway disease (fSAD, or “air trapping”), emphysema, and parenchymal disease characteristic of infection (or “opacities”). PRM is described in further detail in “Parametric Response Mapping as an Indicator of Bronchiolitis Obliterans Syndrome after Hematopoietic Stem Cell Transplantation” authored by Galbán et al., Biol Blood Marrow Transplant 20 (2014) 1592-1598.

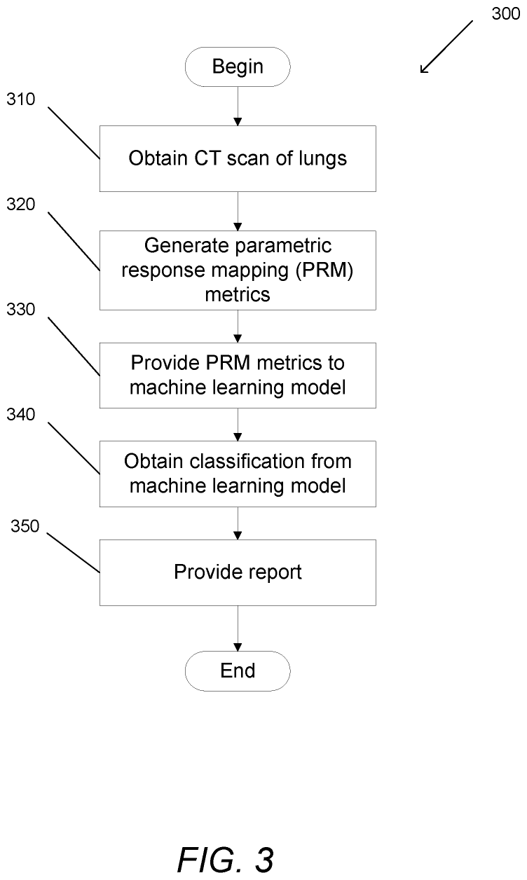

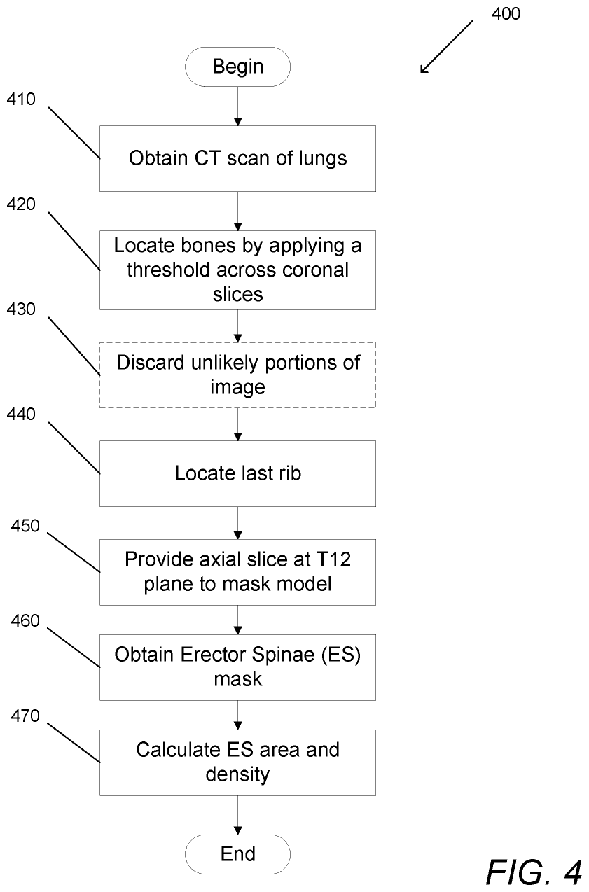

Turning now to FIG. 3, a flow chart for a pulmonary condition identification process in accordance with an embodiment of the invention is illustrated. Process 300 includes obtaining (310) a CT scan of a patient's lungs as imaging data. PRM metrics are generated (320) based on the imaging data. The PRM metrics are provided (330) to a machine learning model. In many embodiments, the machine learning model is an SVM which has been trained using a training data set that includes many sets of PRM metrics derived from CT scans that are annotated with the true condition of the patient. In numerous embodiments, a measurement of the cross-sectional area and/or the density (i.e. percentage of skeletal muscle versus fat) of the erector spinae (ES) at the T12 vertebra can be provided to the machine learning model in order to enhance accuracy. Processes for obtaining these measurements are discussed with respect to FIG. 4 further below. In a variety of embodiments, the total pulmonary vascular volume can be provided to the machine learning model in order to enhance accuracy. In this case, the training data set also includes the cross-sectional area, density, and/or total pulmonary vascular volume metrics for each record in the data set depending on the inputs to be provided to the trained model.

A classification of the patient is obtained (340) from the machine learning model that identifies whether or not the patient is suffering from a pulmonary condition. In many embodiments, the pulmonary condition is BOS, however depending on the training data, different classifications are possible. A report containing the identification is then provided (350). In many embodiments, the report contains a confidence metric indicating a probability of correct classification.

Turning now to FIG. 4, an automated process for deriving the cross-sectional area and density of the ES in accordance with an embodiment of the invention is illustrated. In many embodiments, the cross-sectional area and density of the ES is determined at the plane of the T12 vertebra. Process 400 includes obtaining (410) a CT scan of the patient's lungs. Bones in the CT scan are identified by applying a threshold to the HU unit value across coronal slices of the CT scan. In numerous embodiments, the spine is specifically identified as the center of a peak in HU units across coronal slices. In various embodiments, sections of the CT scan that are unlikely to contain the T12 vertebra are discarded (430). It can be assumed that the T12 vertebra is roughly coplanar with the bottom-most rib. Therefore, approximately the top 50% of the CT scan can be easily discarded. Similarly, the portion of the CT scan identified as containing the spine can be discarded.

The bottom-most rib is located (440). In many embodiments, the bottom-most rib is simply identified by being the lowest horizontal bone structure. However, machine learning models and/or other image processing techniques can be tried to identify the bottom-most rib as well. An axial slice of the CT scan at the identified location of the T12 vertebra is provided (450) to a mask model. In many embodiments, the mask model is a machine learning model trained to identify the ES muscle in axial CT slides at the T12 vertebra. A mask is obtained (460) from the model which defines the portion of the axial slice that contains the ES. Using the mask and the axial slice, the cross-sectional area of the ES can be calculated (470) (i.e. the total area of the mask), and the density of the ES can be calculated (470) by averaging the HU values for voxels within the mask. As can be readily appreciated, any number of image processing techniques can be used to identify the ES, including (but not limited to) those that use neural networks, and/or any other machine learning modality as appropriate to the requirements of specific applications of embodiments of the invention. Further, similar results may be achieved using cross-sectional area and density of the ES at locations other than T12 without departing from the scope or spirit of the invention.

Although specific systems and methods for identification of pulmonary conditions are discussed above, many different systems and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method for identifying pulmonary conditions in hematopoietic cell transplantation patients, comprising:
obtaining a computed tomography (CT) scan of a patient's lungs;
calculating a plurality of parametric response mapping (PRM) metrics;
providing the plurality of PRM metrics to a machine learning model;
obtaining a classification of the CT scan as indicating whether or not the patient's lungs present with a pulmonary condition; and
providing a report comprising the classification.

2. The method for identifying pulmonary conditions in hematopoietic cell transplantation patients of claim 1, wherein the PRM metrics comprise classifications of voxels in the CT scan as presenting with one of: normal lung parenchyma, functional small airway disease, emphysema, and parenchymal disease.

3. The method for identifying pulmonary conditions in hematopoietic cell transplantation patients of claim 1, further comprising providing at least one metric describing an erector spinae muscle of the patient (ES) to the machine learning model.

4. The method for identifying pulmonary conditions in hematopoietic cell transplantation patients of claim 3, wherein the at least one metric describing the ES is calculated by:
locating bones within coronal slices of the CT scan;
identifying a coronal slice of the CT scan that contains an image of a spine of the patient;
locating the bottom-most rib of the patient in the coronal slice;
providing an axial slice of the CT scan at the location of the bottom-most rib to a mask model; and
obtaining a mask describing the area of the axial slice that contains an image of the ES from the mask model.

5. The method for identifying pulmonary conditions in hematopoietic cell transplantation patients of claim 4, wherein the at least one metric is cross-sectional area of the ES, and the method further comprising calculating the cross-sectional area of the ES as the area of the axial slice containing the image of the ES according to the mask.

6. The method for identifying pulmonary conditions in hematopoietic cell transplantation patients of claim 4, wherein the at least one metric is density of the ES, and the method further comprising calculating the density as the average Housenfield unit value of voxels in the axial slice containing the image of the ES according to the mask.

7. The method for identifying pulmonary conditions in hematopoietic cell transplantation patients of claim 4, further comprising discarding the top half of the CT scan.

8. The method for identifying pulmonary conditions in hematopoietic cell transplantation patients of claim 1, wherein the machine learning model is a support vector machine.

9. The method for identifying pulmonary conditions in hematopoietic cell transplantation patients of claim 1, wherein the pulmonary condition is bronchiolitis obliterans syndrome.

10. A device for identifying pulmonary conditions in hematopoietic cell transplantation patients, comprising:
a processor; and
a memory, the memory containing a pulmonary condition identification application directs the processor to:
obtain a computed tomography (CT) scan of a patient's lungs;
calculate a plurality of parametric response mapping (PRM) metrics;
provide the plurality of PRM metrics to a machine learning model;
obtain a classification of the CT scan as indicating whether or not the patient's lungs present with a pulmonary condition; and
provide a report comprising the classification.

11. The device for identifying pulmonary conditions in hematopoietic cell transplantation patients of claim 10, wherein the PRM metrics comprise classifications of voxels in the CT scan as presenting with one of: normal lung parenchyma, functional small airway disease, emphysema, and parenchymal disease.

12. The device for identifying pulmonary conditions in hematopoietic cell transplantation patients of claim 10, wherein the pulmonary condition identification application further directs the processor to provide at least one metric describing an erector spinae muscle of the patient (ES) to the machine learning model.

13. The device for identifying pulmonary conditions in hematopoietic cell transplantation patients of claim 12, wherein in order to calculate the at least one metric describing the ES, the pulmonary condition identification application further directs the processor to:
locate bones within coronal slices of the CT scan;
identify a coronal slice of the CT scan that contains an image of a spine of the patient;
locate the bottom-most rib of the patient in the coronal slice;
provide an axial slice of the CT scan at the location of the bottom-most rib to a mask model; and
obtain a mask describing the area of the axial slice that contains an image of the ES from the mask model.

14. The device for identifying pulmonary conditions in hematopoietic cell transplantation patients of claim 13, wherein the at least one metric is cross-sectional area of the ES, and the pulmonary condition identification application further directs the processor to calculate the cross-sectional area of the ES as the area of the axial slice containing the image of the ES according to the mask.

15. The device for identifying pulmonary conditions in hematopoietic cell transplantation patients of claim 13, wherein the at least one metric is density of the ES, and the pulmonary condition identification application further directs the processor to calculate the density of the ES as the average Housenfield unit value of voxels in the axial slice containing the image of the ES according to the mask.

16. The device for identifying pulmonary conditions in hematopoietic cell transplantation patients of claim 13, wherein the pulmonary condition identification application further directs the processor to discard the top half of the CT scan.

17. The device for identifying pulmonary conditions in hematopoietic cell transplantation patients of claim 10, wherein the machine learning model is a support vector machine.

18. The device for identifying pulmonary conditions in hematopoietic cell transplantation patients of claim 10, wherein the pulmonary condition is bronchiolitis obliterans syndrome.

19. A method for identifying pulmonary conditions in hematopoietic cell transplantation patients, comprising:
obtaining a computed tomography (CT) scan of a patient's lungs;
calculating a plurality of parametric response mapping (PRM) metrics;
calculating a density and a cross-sectional area of an erector spinae muscle of the patient at a level of a T12 vertebra of the patient;
providing the plurality of PRM metrics, the density, and the cross-sectional area to a machine learning model;
obtaining a classification of the CT scan as indicating whether or not the patient's lungs present with a pulmonary condition; and
providing a report comprising the classification.

20. The method for identifying pulmonary conditions in hematopoietic cell transplantation patients of claim 19, further comprising:
locating bones within coronal slices of the CT scan;
identifying a coronal slice of the CT scan that contains an image of a spine of the patient;
locating the bottom-most rib of the patient in the coronal slice;
providing an axial slice of the CT scan at the location of the bottom-most rib to a mask model; and
obtaining a mask describing the area of the axial slice that contains an image of the erector spinae from the mask model;
calculating the cross-sectional area of the erector spinae muscle as the area of the axial slice containing the erector spinae according to the mask; and
calculating the density of the erector spinae muscle as the average Housenfield unit value of voxels in the axial slice containing the erector spinae according to the mask.
